(19) United States
(12) Patent Application Publication
(10) Pub. No.: US 2016/0000404 A1
(43) Pub. Date: Jan. 7, 2016

(54) ULTRASOUND DIAGNOSIS APPARATUS

(71) Applicants: Kabushiki Kaisha Toshiba, Minato-ku (JP); Toshiba Medical Systems Corporation, Otawara-shi (JP)

(72) Inventor: Mio UTSUNOMIYA, Nasushiobara (JP)

(73) Assignees: Kabushiki Kaisha Toshiba, Minato-ku (JP); Toshiba Medical Systems Corporation, Otawara-shi (JP)

(21) Appl. No.: 14/792,128

(22) Filed: Jul. 6, 2015

(30) Foreign Application Priority Data
Jul. 4, 2014 (JP) 2014-139057

(51) Int. Cl. A61B 8/14; A61B 8/00; A61B 8/08

(52) U.S. Cl. CPC A61B 8/14; A61B 8/5207; A61B 8/4494; A61B 8/5223

(57) ABSTRACT

According to one embodiment, an ultrasound diagnosis apparatus includes an ultrasound probe, a calculator, a transmission and reception unit, and a medical image generator. The ultrasound probe includes an ultrasound probe configured to transmit and receive ultrasound waves to perform a scan in a first direction and a second direction perpendicular to the first direction. The calculator calculates scan line densities each in the first direction and the second direction based on parameters as conditions for imaging. The transmission and reception unit drives the ultrasound transducer to transmit and receive ultrasound waves so that the scan line densities are at a predetermined ratio. The medical image generator generates a medical image based on the scan lines received.

### CROSS-REFERENCE TO RELATED APPLICATION

This application is based upon and claims the benefit of priority from Japanese Patent Application No. 2014-139057, filed Jul. 4, 2014; the entire contents of which are incorporated herein by reference.

### FIELD

Embodiments described herein relate generally to an ultrasound diagnosis apparatus.

### BACKGROUND

An ultrasound diagnosis apparatus transmits ultrasound waves to a subject and receives reflected waves therefrom via an ultrasound probe, and converts the amplitude of the reflected waves into the brightness to thereby generate a two-dimensional tomographic image of the subject. In a volume scan, the ultrasound diagnosis apparatus can obtain a plurality of two-dimensional tomographic images of spatially different positions by performing the scan while moving the ultrasound probe in a direction perpendicular to the images. These two-dimensional tomographic images are arranged three-dimensionally to be displayed on the ultrasound diagnosis apparatus as a three-dimensional image.

The volume scan requires the setting of various parameters including scanning conditions such as, scan angle, swing angle, and volume rate, and transmit/receive conditions such as, scanning depth, focus, pulse repetition frequency (prf), transmission frequency, and sound field settings. These parameters such as, scanning conditions and transmit/receive conditions are stored (set) in advance in the ultrasound diagnosis apparatus, and retrieved for use in scanning a subject.

### BRIEF DESCRIPTION OF THE DRAWINGS

FIG. 1 is a block diagram illustrating the internal structure of an ultrasound diagnosis apparatus according to an embodiment;

FIG. 2 is a block diagram illustrating the internal structure of a calculator of the ultrasound diagnosis apparatus;

FIG. 3 is a flowchart of the operation of the ultrasound diagnosis apparatus for calculating a raster count and the number of planes to generate a medical image;

FIG. 4 is a diagram for explaining the concept of the raster count and the number of planes;

FIG. 5 is a diagram for explaining that each site to be scanned requires different volume data;

FIG. 6 is a diagram for explaining that each site to be scanned requires different volume data;

FIG. 7 is a view of an example of a medical image displayed on a display of the ultrasound diagnosis apparatus;

FIG. 8 is a view of an example of a medical image generated without the raster count and the number of planes calculated in the embodiment;

FIG. 9 is a flowchart of the operation of the ultrasound diagnosis apparatus for calculating a raster count and the number of planes to generate a medical image upon setting the ratio of the raster density and the plane density to 1:1;

FIG. 10 is a view of an example of a medical image generated using the raster count and the number of planes calculated in the embodiment;

FIG. 11 is a flowchart of the operation of the ultrasound diagnosis apparatus for calculating a raster count and the number of planes to generate a medical image by another method;

FIG. 12 is a view of an example of a medical image generated through the flow illustrated in FIG. 11;

FIG. 13 is a flowchart of the operation of the ultrasound diagnosis apparatus for calculating a raster count and the number of planes to generate a medical image by still another method;

FIG. 14 is a flowchart of the operation of the ultrasound diagnosis apparatus for calculating a raster count and the number of planes to generate a medical image to maintain the image quality upon changing the volume rate;

FIG. 15 is a view of an example of a medical image generated without the raster count and the number of planes calculated in the embodiment;

FIG. 16 is a view of an example of a medical image generated using the raster count and the number of planes calculated in the embodiment.

### DETAILED DESCRIPTION

In general, according to one embodiment, an ultrasound diagnosis apparatus includes an ultrasound probe, a calculator, a transmission and reception unit, and a medical image generator. The ultrasound probe includes an ultrasound transducer to transmit/receive ultrasound waves. The calculator calculates a raster count in the scan direction and the number of planes in the swing direction using the raster density of a scan cross section and the plane density of a swing cross section based on parameters as conditions for imaging. The transmission and reception unit drives the ultrasound transducer to transmit and receive scan lines based on the raster count and the number of planes. The medical image generator generates a medical image based on the scan lines received.

Referring now to the drawings, a description is given of an ultrasound diagnosis apparatus according to embodiments.

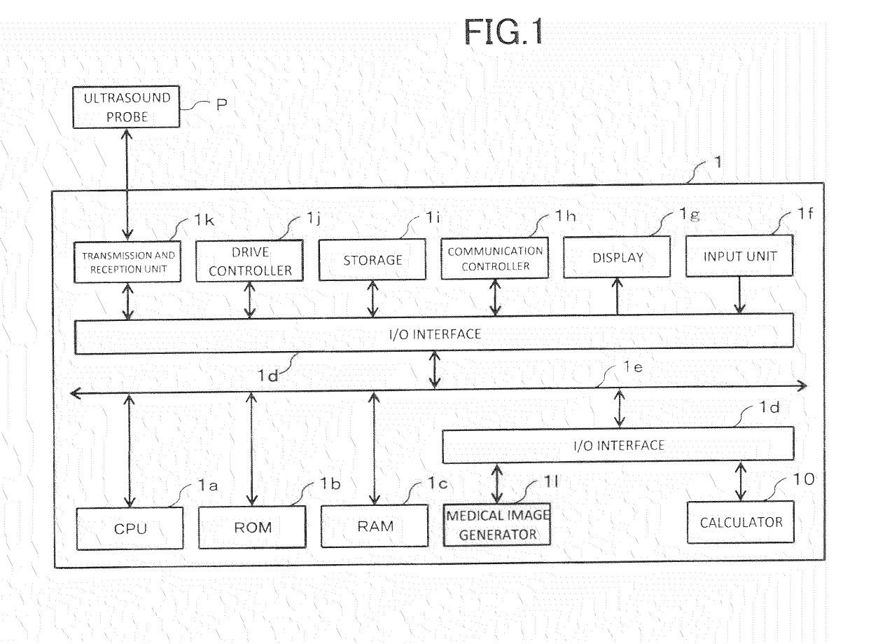

FIG. 1 is a block diagram illustrating the internal structure of an ultrasound diagnosis apparatus 1 according to an embodiment. The ultrasound diagnosis apparatus 1 includes a central processing unit (CPU) 1a, a read only memory (ROM) 1b, a random access memory (RAM) 1c, and an input/output (I/O) interface 1d, which are connected via a bus 1e. The I/O interface 1d is connected to an input unit 1f, a display 1g, a communication controller 1h, and a storage 1i.

The I/O interface 1d is also connected to a drive controller 1j configured to control each drive unit that constitutes the ultrasound diagnosis apparatus 1, a transmission and reception unit 1k configured to transmit/receive ultrasound beams to/from a subject through an ultrasound prove P, and a medical image generator 11 configured to generate a medical image based on the ultrasound beams tranceived.

The I/O interface 1d is further connected to a calculator 10 configured to calculate a raster count and the number of planes required to scan a subject.

In response to an input signal from the input unit 1f, the CPU 1a loads a boot program for booting the ultrasound diagnosis apparatus 1 from the ROM 1b and executed it, and also reads various operating systems from the storage 1i. Besides, the CPU 1a controls various devices based on input signals received through the input unit 1f and the I/O interface 1d from external devices (not illustrated).

The CPU 1a is a processor that loads programs and data stored in the RAM 1c, the storage 1i, and the like into the RAM 1c to implement a series of processes such as filtering based on commands of the programs read out from the RAM 1c.

The input unit 1f includes input devices such as dials and a keyboard used to provide various inputs by medical staff as the operator of the ultrasound diagnosis apparatus 1. The input unit 1f generates an input signal based on operator's input, and sends it to the CPU 1a via the bus 1e. The ultrasound diagnosis apparatus 1 may further include a dedicated operation panel in addition to the keyboard and the like.

The display 1g may be a liquid crystal display (LCD), and receives an output signal from, for example, the CPU 1a via the bus 1e. The display 1g displays a medical image generated based on data acquired through the ultrasound prove P, processing results obtained by the CPU 1a, and the like.

The communication controller 1h may be, for example, a LAN card or a modem, and enables the ultrasound diagnosis apparatus 1 to connect to a communication network such as the Internet or LAN. The data exchanged with the communication network via the communication controller 1h is sent/received through the I/O interface 1d and the bus 1e to/from the CPU 1a as an input or output signal.

The storage 1i includes a semiconductor memory or a magnetic disk, and stores data and programs to be executed by the CPU 1a. For example, the storage 1i stores various types of conditions set to scan a subject by the ultrasound diagnosis apparatus 1, such as scanning conditions and transmit/receive conditions. The storage 1i also stores arithmetic expressions to calculate a raster count and the number of planes based on the conditions.

Besides, the ultrasound diagnosis apparatus 1 of the embodiment stores an ultrasound image generation program in, for example, the storage 1i. The CPU 1a loads and executes the program, thereby implementing the functions of the ultrasound diagnosis apparatus 1.

The transmission and reception unit 1k performs ultrasound scan on a subject through the ultrasound probe P (described later) under the control of the CPU 1a. Examples of the ultrasound scan include B-mode scan, color Doppler mode scan, Doppler mode scan, and the like. Although not illustrated in FIG. 1, the transmission and reception unit 1k includes therein, for example, a preamplifier, an analog-to-digital converter, a receiving delay circuit, an adder, and the like.

The ultrasound probe P is brought in direct contact with a subject to acquire information on the inside of the subject (internal information) by the reflection of ultrasound waves, and is connected to the transmission and reception unit 1k. The ultrasound probe P includes an ultrasound transducer to transmit/receive ultrasound waves. In response to a command from the transmission and reception unit 1k, the ultrasound transducer is driven. The transmission and reception unit 1k receives the internal information on the subject collected by the ultrasound probe P in the form of ultrasound beams, and sends it to the medical image generator 11.

Incidentally, the ultrasound probe P may be, for example, a mechanical 4D probe or a matrix array probe.

Although not illustrated in FIG. 1, the medical image generator 11 includes therein a signal processor, an image composer, and an image generator, and generates a medical image based on information acquired through the ultrasound probe P and the transmission and reception unit 1k.

The signal processor performs various types of signal processing based on a signal received by a receiver of the transmission and reception unit 1k. Specifically, the signal processor performs signal processing according to the scam mode as described above. The signal processor includes therein, for example, an echo data detector, a logarithmic compressor, and digital filters for depth, scan line, and frame directions.

The image composer serves as a scan converter that, having received a signal sent from the signal processor, converts the coordinates of beam data aligned in the depth direction by the signal processor into pixel data aligned in the line direction for display. This processing generates image data to be a base for generating an ultrasound image.

The image generator generates a medical image displayed as a two-dimensional image, for example, based on the image data generated by the image composer. In addition, the image generator generates a three-dimensional medical image based on such two-dimensional medical images.

The calculator 10 calculates a raster count and the number of planes required to scan a subject by the ultrasound diagnosis apparatus 1. The calculator 10 calculates a raster count and the number of planes based on the raster density and the plane density. Further details are described later.

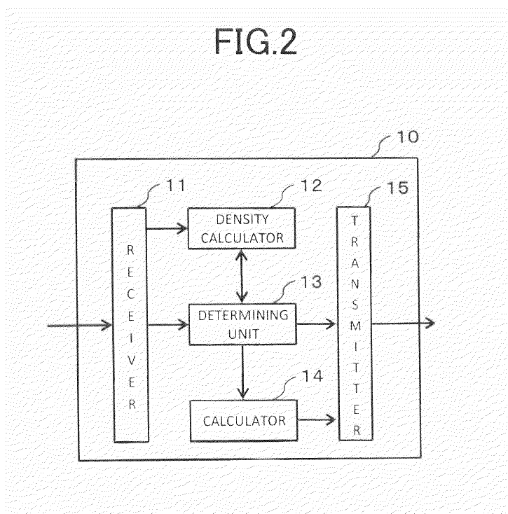

FIG. 2 is a block diagram illustrating the internal structure of the calculator 10 of the embodiment. The calculator 10 includes a receiver 11, a density calculator 12, a determining unit 13, a calculator 14, and a transmitter 15. The receiver 11 receives a processing request as a trigger to calculate a raster count and the number of planes. The density calculator 12 calculates the raster density and the plane density. The calculator 14 calculates a raster count and the number of planes using the raster density and the plane density. The transmitter 15 transmits the raster count and the number of planes to, for example, the CPU 1a so that they are available as scanning conditions.

The function and operation of each unit are described later in the flow of obtaining a raster count and the number of planes to scan a subject based on these conditions and generating a medical image.

In the following, a description is given of the calculation of a raster count and the number of planes required to scan a subject by the ultrasound diagnosis apparatus 1 and the flow of generating a medical image based on a scan performed using them as conditions.

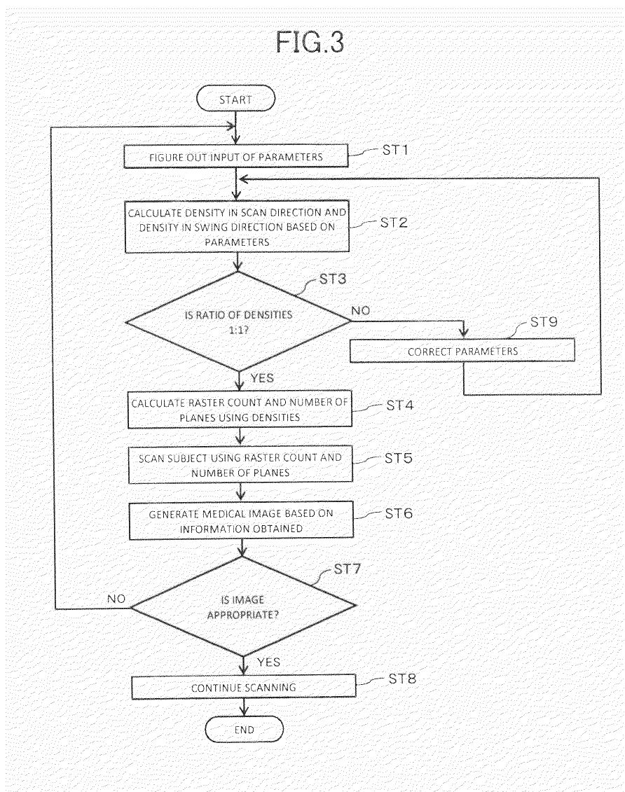

FIG. 3 is a flowchart of the operation of the ultrasound diagnosis apparatus 1 for calculating a raster count and the number of planes to generate a medical image. It is assumed herein that a mechanical 4D probe is used as the ultrasound probe P.

The medical staff who uses the ultrasound diagnosis apparatus 1 enters various conditions (parameters) such as scanning conditions and transmit/receive conditions for a scan before scanning a subject. The ultrasound diagnosis apparatus 1 figures out the contents of information related to the input parameters (ST1).

Here, the medical staff may enter all the parameters, or may use or change parameters stored in the storage 1i to set the parameters.

The medical staff sets the parameters using, for example, the keyboard or the like of the input unit 1f. The contents of the input are displayed on the display 1g. When the medical staff starts entering the parameters, the ultrasound diagnosis apparatus 1 understands this fact and informs beforehand the calculator 10 of that the parameters are to be entered.

Upon receipt of the parameters having been entered and decided, the calculator 10 calculates density in the scan direction and density in the swing direction based on the parameters (ST2). The density in the scan direction is the raster density, and the density in the swing direction is the plane density.

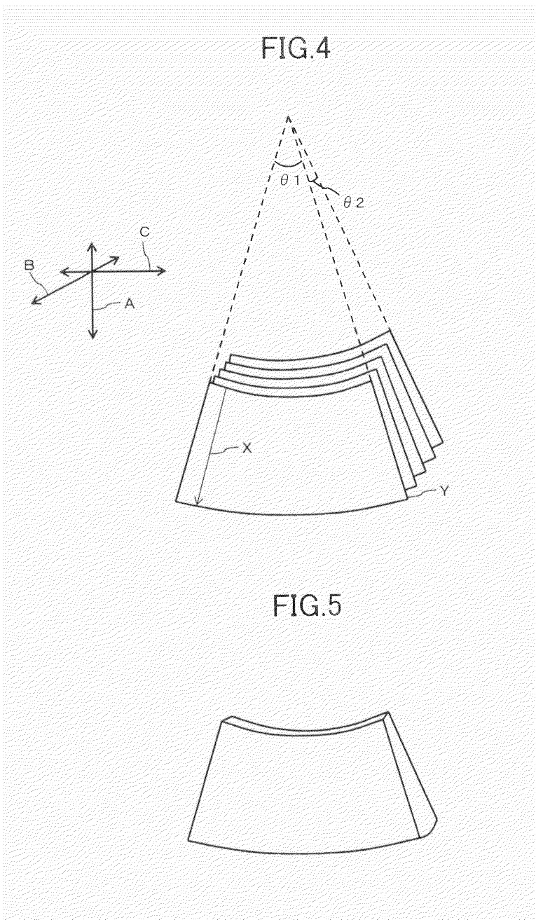

In the following, the meaning of the words such as raster (scan line) and plane (tomographic image) is explained with reference to the drawings. FIG. 4 is a diagram for explaining the concept of the raster count and the number of planes. In FIG. 4, the substantially trapezoidal area having arc-like upper and lower edges is an area to be scanned by the ultrasound diagnosis apparatus 1.

The solid arrow X in the area indicates a raster (scan line). A plane Y is represented as a single medical image generated by performing transmission/receipt of waves such that the raster X forms the scan area in the range of scan angle θ1. That is, in FIG. 4, the substantially trapezoid Y represents a plane (tomographic image). The raster count is the number of rasters X to be transmitted/received to generate a single plane Y.

By shifting the ultrasound probe P little by little in scanning, a plurality of planes Y can be obtained. The swing angle refers to the angle at which the ultrasound probe P is shifted, and is indicated by reference sign θ2 in FIG. 4. The acquisition of sufficient amount of volume data to generate a plurality of planes Y within the range of the swing angle θ2 enables the generation of a three-dimensional medical image. Here, the number of planes indicates the number of planes Y generated in the swing angle θ2.

With a scan performed in this manner, it is possible to acquire the volume data of a site of a subject to be examined. Note that the shape of the volume data to be acquired may vary depending on the site to be examined.

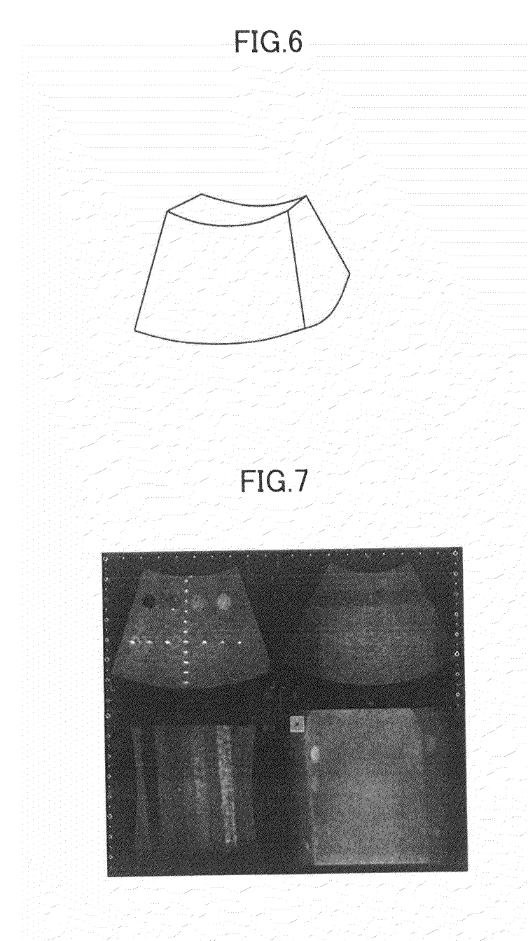

FIGS. 5 and 6 are diagrams for explaining that each site to be scanned requires different volume data.

For example, if the uterus or the backbone of a fetus is an object to be examined, required volume data has a wide-thin shape as illustrated in FIG. 5. Accordingly, the volume data is acquired with the scan angle θ1 larger than the swing angle θ2.

On the other hand, if the heart of a fetus is an object to be examined, required volume data is in a shape having a width and a depth similar to each other as illustrated in FIG. 6. Accordingly, the volume data is acquired with the scan angle θ1 substantially the same as the swing angle θ2.

The volume data thus acquired is displayed on the display 1g of the ultrasound diagnosis apparatus 1. FIG. 7 is a view of an example of a medical image displayed on the display 1g of the ultrasound diagnosis apparatus 1.

As illustrated in FIG. 7, the screen of the display 1g is divided into four display areas and displays four types of images. Among them, an image displayed in the lower right area is a volume image acquired. Images displayed in other three areas are tomographic images obtained by slicing the volume image into cross sections.

In FIG. 4 that illustrates the raster X and the planes Y, A indicates the direction of the raster X as the scan direction, and B indicates the direction perpendicular to the scan direction A, in which the planes Y are captured, that is, the swing direction. Besides, C indicates a direction perpendicular to the scan direction A as well as the swing direction B.

In FIG. 7, the display 1g displays tomographic images obtained by slicing the volume image in the respective directions into cross sections. Specifically, in FIG. 7, the display 1g displays a tomographic image (plane A) in the scan direction A in the upper left area, a tomographic image (plane B) in the swing direction B in the upper right area, and a tomographic image (plane C) in the C direction in the lower left area.

Note that medical staff who uses the ultrasound diagnosis apparatus 1 may arbitrarily set the layout of the display areas on the display 1g including the size of each area and what type of image is displayed in each area.

The density calculator 12 calculates the density of the rasters X (raster density) in the scan direction A and the density of the planes Y (plane density) in the swing direction B, and sends the calculation results to the determination unit 13. The determination unit 13 determines whether the ratio of the raster density and the plane density is 1:1 (ST3). In other words, the ratio of the raster density and the plane density is adjusted to 1:1 to thereby improve the image quality of display images.

Having determined that the ratio of the raster density and the plane density is 1:1 (YES in step ST3), the determination unit 13 sends the determination result to the calculator 14. The calculator 14 calculates a raster count and the number of planes using the densities (ST4). The calculator 14 calculates the time required to acquire a piece of volume data in volume scanning as well as a raster count and the number of planes using the following expression 1 as a reference expression:

$$[ Expression 1 ]$$

$$\{ \begin{matrix}T = ( {tX}_{1} ) {X}_{2} + α \\ {X}_{1} = {θ}_{1} {k}_{1} f ( {i}_{1} , {j}_{1} , {l}_{1} ⋯ ) \\ {X}_{2} = {θ}_{2} {k}_{2} f ( {i}_{2} , {j}_{2} , {l}_{2} ⋯ )\end{matrix} \qquad ( 1 )$$

where X1 represents the raster count in the scan direction, X2 represents the number of planes in the swing direction B, t represents pulse transmission interval, and a represents the time anticipated to be necessary for the switching of the swing direction and the like. Thereby, the time required to acquire a piece of volume data in volume scanning can be calculated. The raster count X1 and the number of planes X2 are calculated using corresponding equations in the equations (1).

In the embodiment, the raster count X1 or the number of planes X2 is calculated using raster density k1 or plane density k2.

Here, θ1 represents the scan angle, θ2 represents the swing angle, k1 represents the raster density, k2 represents the plane density, and f(y) represents a correction function. The correction function f(y) is intended to make a correction in consideration of the presence of processing such as compound processing that is unable to accurately express a raster count by only a volume rate and prf in the process of generating and displaying volume data.

With the raster count X and the number of planes X2 thus calculated, a scan is performed on the subject (ST5). As a result of the scan, the medical image generator 11 generates a volume image as the medical image (ST6), and the display 1g displays it. In addition to the volume image, the display 1g displays cross sections obtained by slicing the volume image into the planes A, B and C as illustrated in FIG. 7.

While viewing the medical image, the medical staff as the operator checks whether the display image is the intended one. In other words, the medical staff determines whether the display image is appropriate, and also whether the setting of the parameters (raster density, plane density) related to the image generation is appropriate. For example, the ultrasound diagnosis apparatus 1 displays a confirmation screen such as a pop-up window or the like asking whether the image is appropriate to allow the medical staff to determine it. Thus, it is determined whether the display image is appropriate (ST7).

Upon detecting that the medical staff has entered the determination on the confirmation screen, the ultrasound diagnosis apparatus 1 determines whether the display image is appropriate. When the display image is appropriate (YES in step ST7), the scan is continued without changing the conditions (ST8). On the other hand, if the display image is not appropriate (NO in step ST7), the parameters are set again, and the raster density and the plane density are calculated again.

As mentioned above, FIG. 7 illustrates an example of a medical image displayed on the display 1g of the ultrasound diagnosis apparatus 1. Regarding the tomographic images in FIG. 7, the ratio of the raster density and the plane density is about 1:1. Accordingly, in this example, the tomographic image of the plane A in the upper left area and the tomographic image of the plane B in the upper right area are displayed with excellent image quality. In addition, since the tomographic images of the planes A and B have a high image quality, the both are displayed with balance in the view of the entire screen.

On the other hand, if the determination unit 13 determines that the ratio of the raster density and the plane density is not 1:1 (NO in ST3), it is determined that a medical image of a high image quality cannot be achieved in this state.

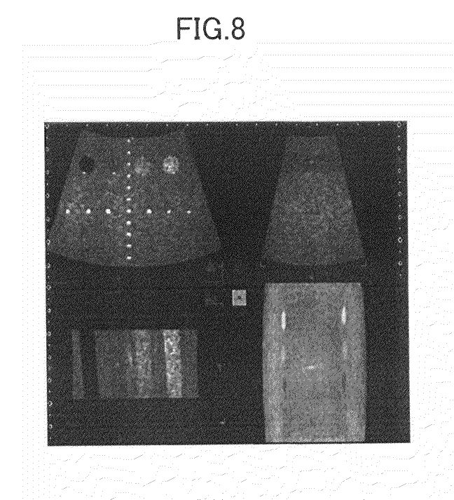

FIG. 8 is a view of an example of a medical image generated without the raster count and the number of planes calculated in the embodiment. Namely, FIG. 8 illustrates an example in which there is a difference in image quality between the tomographic image of the plane A and the tomographic image of the plane B.

In the example of FIG. 8, the plane density is higher than the raster density, and thus the resolution of the plane B is higher than that of the plane A. If the ratio of the raster density and the plane density is out of balance differently from the display example illustrated in FIG. 7, for example, while the tomographic image of the plane B is clearly displayed, the tomographic image of the plane A is displayed with poor image quality. The image of either one of the planes may have an increased image quality. However, considering that tomographic images are generally generated with respect to a required area in a volume image, images can be more easily viewable when at least the tomographic images of the planes A and B have about the same image quality.

The viewability is achieved by adjusting the ratio of the raster density and the plane density to be balanced, to nearly 1:1, so that display images are obtained by a scan performed with the appropriate setting of a raster count and the number of planes. For this reason, the parameters are corrected such that the ratio of the raster density and the plane density becomes 1:1 in the calculator 10 of the ultrasound diagnosis apparatus 1 (ST9). As a result, the ratio of the raster density and the plane density is set to 1:1. Note that, even when the correction is made automatically, parameters entered by the medical staff or the like are not fundamentally corrected.

A description has been given above of the process to approximate the ratio of the raster density and the plane density to 1:1 by correcting the parameters as appropriate to thereby solve the imbalance between the tomographic images as illustrated in FIG. 8.

Besides, without correcting the parameters, the ratio of the raster density and the plane density may be set automatically to 1:1 in the ultrasound diagnosis apparatus 1 so that a raster count and the number of planes are calculated under this conditions.

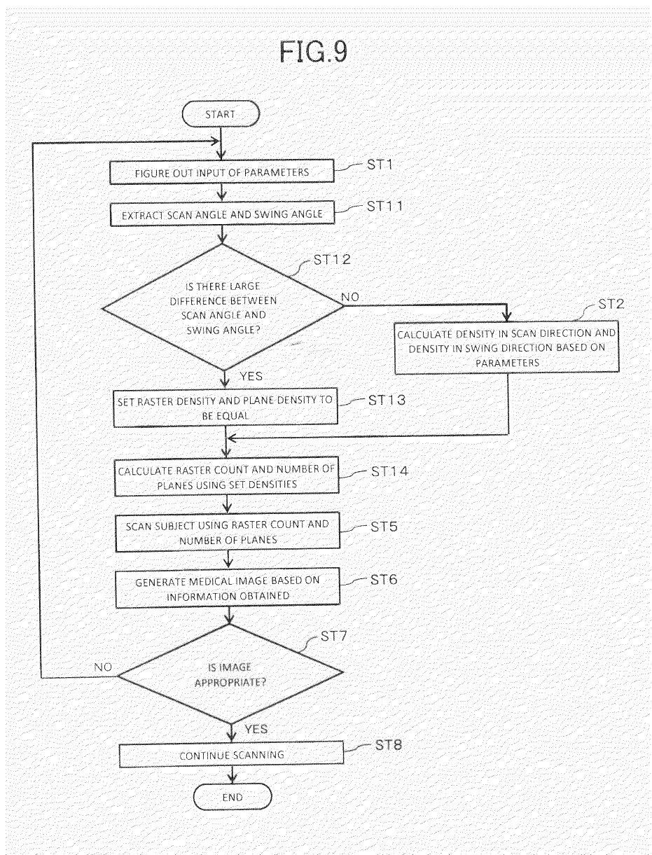

FIG. 9 is a flowchart of the operation of the ultrasound diagnosis apparatus 1 for calculating a raster count and the number of planes to generate a medical image upon setting the ratio of the raster density and the plane density to 1:1. Incidentally, in FIG. 9, like process as in FIG. 3 is denoted by like step number.

First, the ultrasound diagnosis apparatus 1 figures out the values of various parameters (ST1). The determination unit 13 extracts the scan angle and the swing angle from the parameters (ST11), and both the angles are checked (ST12). Here, the scan angle and the swing angle are checked because, if there is a large difference between the two, the values of the raster density and the plane density are separated from each other. This may result in imbalance in image quality between a tomographic image (plane A) in the scan direction and a tomographic image (plane B) in the swing direction.

If the determination unit 13 determines that there is a large difference between the scan angle and the swing angle as a result of checking them (YES in step ST12), the raster density k1 and the plane density k2 are set in advance such that their values are equal (ST13) as represented by the following expression:

[Expression 2]

k1=k2 (2)

If the relationship of expression (2) is reflected in expression (1), the following expression can be obtained:

$$[ Expression 3 ]$$

$$\{ \begin{matrix}{X}_{1} = {\{ {θ}_{1} f ( {i}_{2} , {j}_{2} , {l}_{2} ⋯ ) ( T - α ) \text{/} {θ}_{2} f ( {i}_{1} , {j}_{1} , {l}_{1} ⋯ ) t \}}^{⋀ 1 \text{/} 2} \\ {X}_{2} = {\{ {θ}_{2} f ( {i}_{1} , {j}_{1} , {l}_{1} ⋯ ) ( T - α ) \text{/} {θ}_{1} f ( {i}_{2} , {j}_{2} , {l}_{2} ⋯ ) t \}}^{⋀ 1 \text{/} 2}\end{matrix} \qquad ( 3 )$$

By using expression (3) with the raster density k1 and the plane density k2 set as above, according to scan time T and pulse repetition frequency (prf), the raster count X1 and the number of planes X2 are calculated (ST14). The subject is scanned based on the raster count X1 and the number of planes X2 thus calculated (ST5), and thereby a medical image is generated (ST6). The following processes are performed in the same manner as described above.

In this manner, by automatically setting the raster density k1 and the plane density k2 to k1=k2, it is possible to solve the imbalance between the planes A and B as, for example, illustrated in FIG. 8.

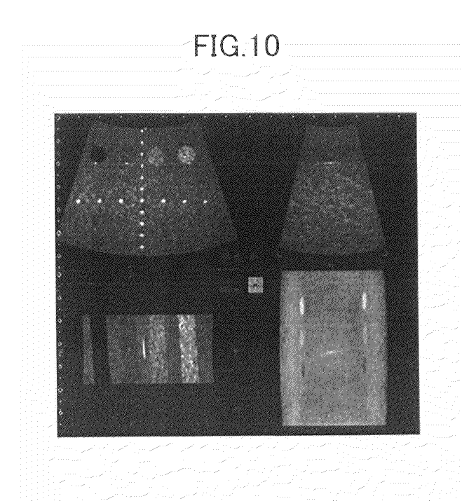

FIG. 10 is a view of an example of a medical image generated using the raster count and the number of planes calculated in the embodiment. Compared to the example of FIG. 8, no imbalance is observed in the qualities of the images of the planes A and B, and the plane A is displayed with the same image quality as that of the plane B. This is because expression (3) takes into account the imbalance between the scan angle and the swing angle.

On the other hand, if the determination unit 13 determines that there is no large difference between the scan angle and the swing angle as a result of checking them (NO in step ST12), the raster density k1 and the plane density k2 are calculated based on the same parameters as entered (ST2), and a raster count and the number of planes are calculated by using the densities (ST14 and subsequent steps).

A description is given of another method of calculating a raster count and the number of planes. This method is performed also using the raster density k1 and the plane density k2 as described above.

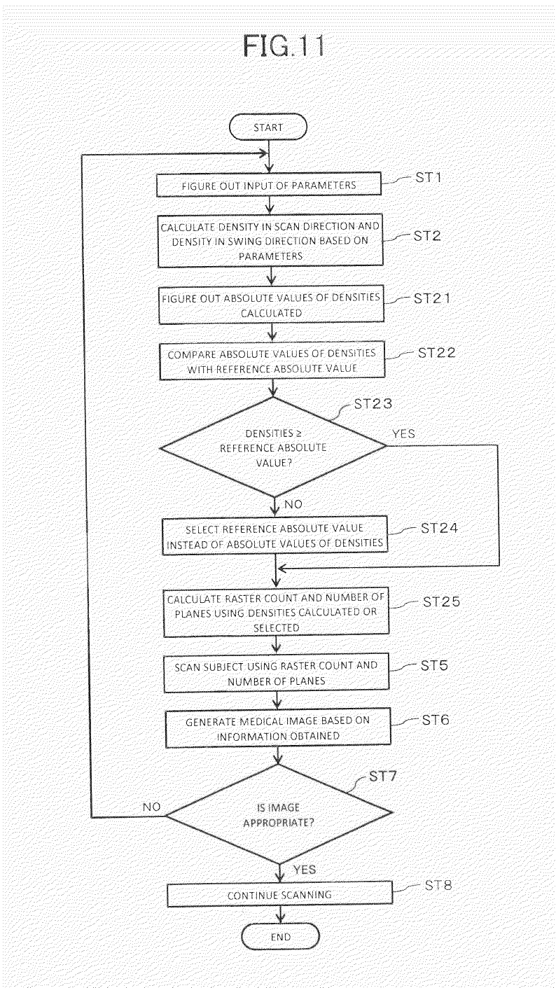

FIG. 11 is a flowchart of the operation of the ultrasound diagnosis apparatus 1 for calculating a raster count and the number of planes to generate a medical image by another method. Incidentally, in the flowchart of FIG. 11, like process as in the precedent flowcharts is denoted by like step number.

Here, appropriate values of a raster count and the number of planes are obtained based on the absolute values of the raster density k1 and the plane density k2 calculated. Described below is the specific flow of calculating a raster count and the number of planes.

First, the ultrasound diagnosis apparatus 1 figures out the values of various parameters set by the medical staff or the like (ST1). The density calculator 12 calculates the raster density and the plane density based on the values (ST2). These steps are performed as described above.

The determination unit 13 receives the values of the raster density and the plane density calculated, and figures out the absolute values of them (ST21). Thereafter, the determination unit 13 compares the absolute values of the both densities with a reference absolute value (ST22). Here, the reference absolute value indicates a value, the use of which as a density for the calculation of a raster count and the number of planes allows the generation of a medical image that maintains appropriate image quality. For example, the reference absolute value indicates a lower limit of the density to maintain the image quality. The reference absolute value is stored in, for example, the storage 1i in advance. The determination unit 13 compares the raster density and the plane density calculated with this reference absolute value.

As a result, when the absolute values of the raster density and the plane density are not equal to or above the reference absolute value (NO in step ST23), the reference absolute value is selected as the values of the densities instead of the absolute values of the raster density and the plane density calculated (ST24).

This is because if a value below the reference absolute value is employed as the raster density or the plane density, the image quality cannot be maintained, which causes, for example, a coarse screen image or imbalance in image quality between the planes A and B. For this reason, to maintain the minimum image quality, the reference absolute value is used as the density for calculating a raster count and the number of planes.

As a result of the comparison between the calculated densities and the reference absolute value, when the values of the calculated densities are equal to or above the reference absolute value (YES in step ST23), a raster count and the number of planes are calculated by using the raster density k1 and the plane density k2 calculated by the density calculator 12.

A raster count and the number of planes are calculated based on the raster density k1 and the plane density k2 calculated or selected by using the above expressions (ST15). Then, the subject is scanned based on the values to obtain volume data. The display 1g displays a volume image and tomographic images of predetermined cross sections based on the volume data (ST5 and subsequent steps).

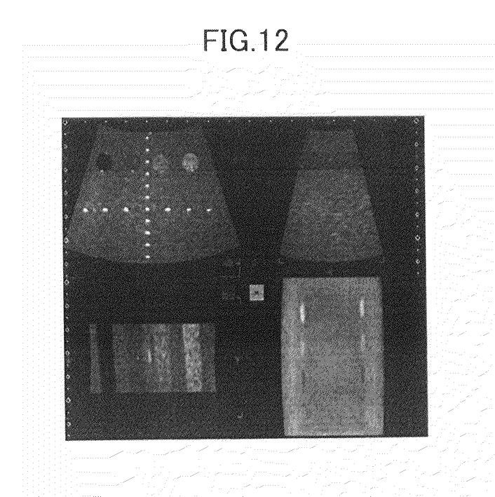

FIG. 12 is a view of an example of a medical image generated through the flow illustrated in FIG. 11. Generally, a higher volume rate makes the screen display coarser, and the image quality degrades. However, by the above processing using the absolute value of the raster density and the plane density, even if the volume rate is increased, the same image quality as with a low volume rate can be maintained.

For example, for the images illustrated in FIGS. 7 and 10, the volume rate is set at 2 vps (volume per second). If a medical image is generated by the same process after the volume rate has been simply increased to, for example, 6 vps, the image quality of the display image degrades.

Nevertheless, as described above, when a raster count and the number of planes are calculated based on the absolute values of the raster density and the plane density to perform a scan, even if the volume rate is increased, as images illustrated in FIG. 12, it is possible to maintain the same image quality as the images of FIGS. 7 and 10 before the increase of the volume rate.

Here, the absolute values of both the raster density and the plane density are compared to the reference absolute value. While this method is advantageous as described above, it does not take into account the balance in image quality between an image of the plane A affected by the raster count and an image of the plane B affected by the number of planes. For example, the following process can prevent the imbalance in image quality between the planes A and B.

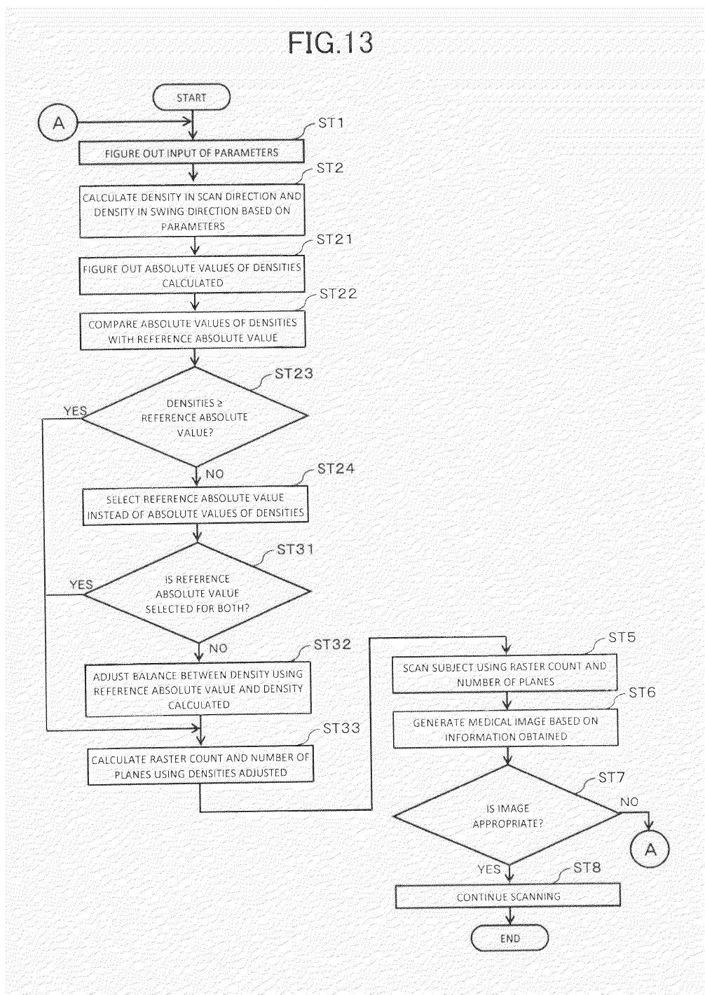

FIG. 13 is a flowchart of the operation of the ultrasound diagnosis apparatus 1 for calculating a raster count and the number of planes to generate a medical image by still another method. This method is characterized in the process performed when the raster density and the plane density are not equal to or above the reference absolute value. Incidentally, in the flowchart of FIG. 13, like process as in the precedent flowcharts is denoted by like step number.

If the raster density and the plane density are less than the reference absolute value, a raster count and the number of planes are calculated using the reference absolute value instead of the densities calculated (ST24). In this case, both the raster density and the plane density may be less than the reference absolute value, or either one of them may be less than the reference absolute value.

When both the raster density and the plane density are less than the reference absolute value, the reference absolute value is selected for the both of them. Therefore, it is unlikely that the densities are imbalanced. On the other hand, if one of them is less than the reference absolute value, while the other is equal to or above the value, an imbalance may occur between the densities.

Accordingly, the determination unit 13 checks whether the absolute values are selected for both the raster density and the plane density (ST31). If the absolute value is selected for either one of the raster density and the plane density (NO in step ST31), both the values are adjusted (ST32). Thus, when a medical image is generated, imbalanced image quality is avoided in the display. Specifically, for example, the following processes are performed.

For example, it is assumed that the absolute value of the raster density k1 is 20, that of the plane density k2 is 8, and the reference absolute value is 10. In this case, the absolute value of the plane density k2 is smaller than the reference absolute value. Thus, by the process as described above, the reference absolute value, 10, is used as the density (k2=10). On the other hand, since the value (20) of the raster density k1 exceeds the reference absolute value, the calculated value is supposed to be used as the raster density k1.

However, it is also possible to employ a process different from the above process that simply uses the calculated value. For example, the calculated value of the plane density k2 may be set as a value of the plane density k2, and the value of the raster density k may be adjusted to maintain the ratio between the raster density k1 calculated and the plane density k2 calculated.

Alternatively, instead of directly using the value of the raster density k1 equal to or above the reference absolute value, considering the value of the plane density k2, another adjustment method may be employed to set the value of the raster density k1 to 18, for example. With this, as well as maintaining the image quality of display images, taking into account the balance in image quality between an image of the plane A affected by the raster count and an image of the plane B affected by the number of planes.

In this manner, if an imbalance may occur in image quality when the absolute value is selected as either one of the raster density or the plane density, the balance of the densities is adjusted, and then a raster count and the number of planes are calculated using the adjusted values (ST33).

On the other hand, if the absolute value is selected for both the raster density and the plane density (YES in step ST31), a raster count and the number of planes are calculated using the values (ST33).

As described above, the values of the raster density k1 and the plane density k2 are determined not simply by the comparison with the reference absolute value. Even when the value of density is equal to or above the reference absolute value, the value is reduced taking into account the balance of the densities. This adjustment can achieve the balance in image quality between an image of the plane A affected by the raster count and an image of the plane B affected by the number of planes.

As has been described above, by setting the ratio of the raster density k1 and the plane density k2 as close as 1:1, it is possible to improve the image quality of an image of the plane A affected by the raster count and an image of the plane B affected by the number of planes. On the other hand, an increase in the volume rate reduces the scan time T per one volume. Accordingly, a sufficient number of raster lines to generate a single volume image cannot be acquired. Therefore, the absolute values of the raster density k1 and the plane density k2 are taken and compared with the reference absolute value. Thus, even when the volume rate is set to a high value, the same image quality as before can be maintained.

The above processes are performed on the assumption that, for example, scan lines are uniformly distributed in the scan direction A or the swing direction B (the density is equalized). However, depending on examinations, there may be a case where it is desired to increase scan lines in a specific direction in, for example, the scan direction A to perform a scan.

For example, when the volume rate is set to a high value, under the condition where the raster density k1 is isotropic, an image of the plane A affected by the raster count and an image of the plane B affected by the number of planes may have a poor image quality unless any measure is taken. Accordingly, sufficient resolution for the diagnosis of medical staff may not be obtained. Therefore, as one measure, the process is performed using the absolute value as described above. This enables the image quality to be maintained and improved under the condition where the raster density k1 is isotropic.

The measure is not limited to this method, and another method may be applicable. Described below is a method intended to maintain and improve the image quality even when, for example, a scan is performed by an increased number of scan lines in a specific direction with anisotropic raster density k1.

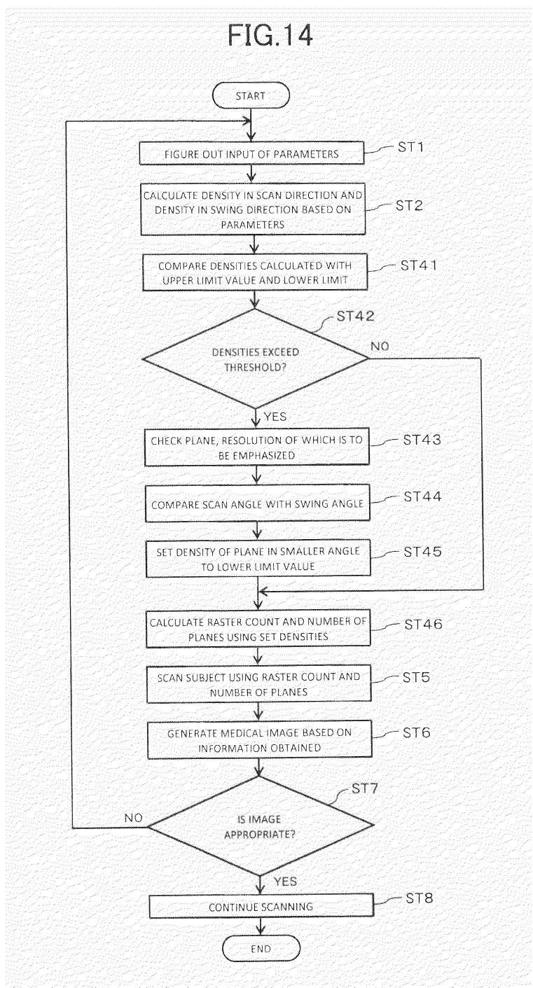

FIG. 14 is a flowchart of the operation of the ultrasound diagnosis apparatus 1 for calculating a raster count and the number of planes to generate a medical image to maintain the image quality upon changing the volume rate. The following description is based on the assumption that not the plane density k2 but the raster density k1 is intentionally changed. Incidentally, in the flowchart of FIG. 14, like process as in the precedent flowcharts is denoted by like step number.

The process described below is characterized in that the upper limit value and the lower limit value of the density are determined in advance, and the raster density k1 and the plane density k2 calculated are each compared with the limit values.

With reference to FIG. 14, first, the ultrasound diagnosis apparatus 1 figures out the values of various parameters set by the medical staff or the like (ST1). The calculator 10 calculates the density (the raster density k1) in the scan direction A and the density (the plane density k2) in the swing direction B using the parameters (ST2).

Next, the determination unit 13 compares the raster density k1 and the plane density k2 calculated by the density calculator 12 with a upper limit value kmax and a lower limit value kmin determined in advance (ST41). For example, the upper limit value kmax and the lower limit value kmin are stored in the storage 1i in advance.

As a result of the comparison, if the values of the raster density k1 and the plane density k2 exceed a threshold, which is the upper limit value kmax or the lower limit value kmin (YES in step ST42), the determination unit 13 checks which of the resolution of a tomographic image (plane A) in the scan direction A and the resolution of a tomographic image (plane B) in the swing direction B is to be emphasized (ST43).

Here, the determination unit 13 makes the determination on which resolution of the plane A or B is to be emphasized by comparing the sizes of the scan angle θ1 and the swing angle θ2 (ST44). That is, a larger angle indicates that the medical staff wishes to view a wider range, and therefore, the image quality is increased for the tomographic image corresponding to the larger angle.

For example, when volume data to be acquired by a scan has a wide-thin shape as illustrated in FIG. 5, the scan angle θ1 is larger than the swing angle θ2. Accordingly, in this case, the medical staff is likely to wish to view the tomographic image of the plane A in the scan direction A rather than the tomographic image of the plane B in the swing direction B. Thus, the process is performed to increase the resolution of the tomographic image of the plane A. In the following, for the convenience of the description, a specific tomographic image, the resolution of which is to be emphasized is referred to as “tomographic image α”, and other tomographic images are referred to as “tomographic image β”.

After the determination unit 13 compares the sizes of the scan angle θ1 and the swing angle θ2, and determines the plane the resolution of which is to be emphasized, the density of the tomographic image β of the plane in a smaller angle is set to the lower limit value kmin (ST45).

The calculator 14 calculates the raster count X1 and the number of planes X2 based on the density set by the determination unit 13 using the following expression (ST46):

$$[ Expression 4 ]$$

$$\{ \begin{matrix}{X}_{1} = f ( {i}_{2} , {j}_{2} , {l}_{2} ⋯ ) ( T - α ) \text{/} {k}_{min} {f ( {i}_{1} , {j}_{1} , {l}_{1} ⋯ )}^{⋀ 2} t \\ {X}_{2} = {k}_{min} {θ}_{2} \text{/} f ( {i}_{2} , {j}_{2} , {l}_{2} ⋯ ) \end{matrix} \qquad ( 4 )$$

By using expression (4), the raster count X1 and the number of planes X2 are obtained based on the density of the tomographic image β of the plane in a smaller angle set as the lower limit value kmin. The subject is scanned using the raster count X1 and the number of planes X2 thus calculated (ST5), and a medical image is generated based on information acquired thereby (ST6). The display 1g displays the medical image. After the medical staff checks the medical image (ST7), the raster density k1 and the plane density k2 are set again, or the scan is continued (ST8).

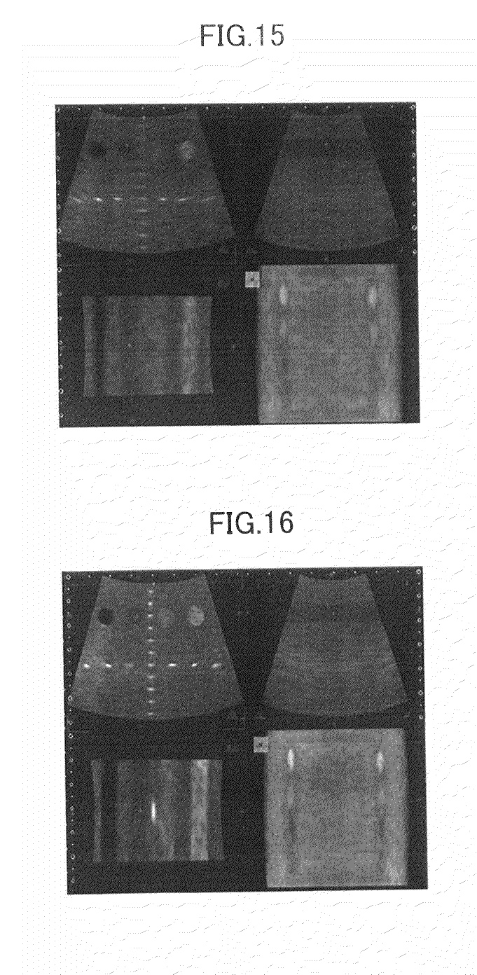

FIG. 15 is a view of an example of a medical image generated without the raster count and the number of planes calculated in the embodiment. FIG. 16 is a view of an example of a medical image generated using the raster count and the number of planes calculated in the embodiment.

As can be seen from the example of FIG. 15, if the volume rate is increased without measure, the image quality of the planes A and B degrades. As a result, resolution necessary for diagnosis cannot be achieved.

On the other hand, when a medical image is generated and displayed using the raster count X1 and the number of planes X2 calculated by expression (4), as can be seen from the example of FIG. 16, resolution is significantly improved in the display image of the plane A as the tomographic image α. This is because the resolution of the plane A is emphasized compared to the plane B as the tomographic image β as described above. Besides, regarding the resolution of the plane B, a higher resolution is achieved compared to the plane B illustrated in FIG. 15, along with the improvement in the resolution of the plane A.

The determination unit 13 is described above as determining which resolution of the plane A or B is to be emphasized by comparing the sizes of the scan angle θ1 and the swing angle θ2. However, the tomographic image α, the resolution of which is to be emphasized, may be selected by, for example, determining in which direction scan lines should be increased to improve the resolution using an image recognition technology.

In addition, the ultrasound diagnosis apparatus 1 may automatically, or based on an instruction from the medical staff, adjust the balance between the density of the tomographic image α, the resolution of which is to be emphasized, and that of the other tomographic image β. With this adjustment, balanced image quality can be achieved on the screen. The medical staff may make the instruction by, for example, setting a plurality of types of patterns each indicating the ratio of the plane density in advance, and selecting one of them.

As described above, as the conditions for performing a volume scan, a raster count and the number of planes are determined based on the raster density and the plane density. Thus, the ultrasound diagnosis apparatus of this embodiment can generate a medical image with improved image quality.

By performing the process of achieving a balance in display tomographic images together, as well as improving the image quality, a medical image is generated and displayed taking into account the balance of display on the entire screen.

The above description has been made on the assumption that the ultrasound probe P used in the ultrasound diagnosis apparatus 1 is a mechanical 4D probe. However, the ultrasound probe P is not limited to the mechanical 4D probe, but may be, for example, a matrix array probe. When a matrix array probe is employed, while the azimuth direction in the case of a matrix array probe corresponds to the scan direction in the case of a mechanical 4D probe, the elevation direction in the case of a matrix array probe corresponds to the swing direction in the case of a mechanical 4D probe. Note that the operation flow and the contents are the same as described above. That is, by setting a raster count and the number of planes using the raster density and the plane density, it is possible to improve the image quality of a medical image generated.

In addition, it has been described that the ultrasound diagnosis apparatus 1 automatically sets a raster count and the number of planes using the raster density and the plane density, and thereby a medical image is generated with improved image quality. However, for example, optimal raster density and plane density for a site to be scanned may be set in advance in the ultrasound diagnosis apparatus 1 with respect to each site. In this case, the medical staff determines a site to be scanned, and, for example, selects an icon of the site displayed on the display screen to set the optimal raster density and plane density or scan angles for scanning the site.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus, comprising:
an ultrasound probe configured to transmit and receive ultrasound waves to perform a scan in a first direction and a second direction perpendicular to the first direction; and
processing circuitry configured to
calculate scan line densities each in the first direction and the second direction based on parameters as conditions for imaging,
transmit and receive ultrasound waves so that the scan line densities are at a predetermined ratio, and
generate a medical image based on the ultrasound waves received.

2. The ultrasound diagnosis apparatus of claim 1, wherein the predetermined ratio is 1:1.

3. The ultrasound diagnosis apparatus of claim 1, wherein the predetermined ratio can be set arbitrarily.

4. The ultrasound diagnosis apparatus of claim 1, wherein the scan in the first direction and the second direction is performed by a two-dimensional array of ultrasound transducers.

5. The ultrasound diagnosis apparatus of claim 1, wherein the scan in the first direction and the second direction is performed by a one-dimensional array of ultrasound transducers by swinging the ultrasound transducers in a direction perpendicular to the array.

6. The ultrasound diagnosis apparatus of claim 1, wherein the processing circuitry is further configured to
calculate raster density and plane density, and
check a ratio between the raster density and the plane density.

7. The ultrasound diagnosis apparatus of claim 6, wherein the processing circuitry is further configured to, when the raster density and the plane density are not equal, set a same value for the raster density and the plane density, and calculate the raster count and the number of planes based on the set value.

8. The ultrasound diagnosis apparatus of claim 6, wherein the processing circuitry is further configured to
figure out an absolute value of the raster density or an absolute value of the plane density upon calculating the raster count or the number of planes,
compare the absolute values with a reference value, and
when the absolute value is less than the reference value, use the reference value instead of the absolute value to obtain the raster count or the number of planes.

9. The ultrasound diagnosis apparatus of claim 8, wherein the processing circuitry is further configured to, when the reference value is selected for one of the raster density and the plane density, and the absolute value is selected for another, adjust balance between the absolute values.

10. The ultrasound diagnosis apparatus of claim 6, wherein the processing circuitry is further configured to
when the raster density and the plane density exceed an upper limit value or a lower limit value determined in advance due to setting of a high volume rate, specify a tomographic image, image quality of which is to be maintained or improved, and
replace density related to a tomographic image other than the tomographic image specified with the lower limit value to set the raster density or the plane density.

11. The ultrasound diagnosis apparatus of claim 10, wherein the processing circuitry is further configured to, when specifying a tomographic image whose image quality is to be maintained or improved, compare a scan angle and a swing angle, and specify a tomographic image corresponding to a plane having larger angle.

12. An ultrasound diagnosis apparatus, comprising:
an ultrasound probe configured to transmit and receive ultrasound waves to perform a scan in a first direction and a second direction perpendicular to the first direction; and
processing circuitry configured to
acquire information related to a site to be scanned, and set scan angels each for the first direction and the second direction based on the information,
transmit and receive ultrasound waves according to the scan angles, and
generate a medical image based on the ultrasound waves received.

13. The ultrasound diagnosis apparatus of claim 12, wherein the scan in the first direction and the second direction is performed by a two-dimensional array of ultrasound transducers.

14. The ultrasound diagnosis apparatus of claim 12, wherein the scan in the first direction and the second direction is performed by a one-dimensional array of ultrasound transducers by swinging the ultrasound transducers in a direction perpendicular to the array.
